(19) United States
(12) Patent Application Publication
(10) Pub. No.: US 2006/0280777 A1
(43) Pub. Date: Dec. 14, 2006

(54) Encapsulated energy gel compositions

(76) Inventor: Andrew Schydlowsky, Santa Barbara, CA (US)

Correspondence Address: FISH & RICHARDSON P.C., PO BOX 1022, MINNEAPOLIS, MN, 55440-1022, US

(21) Appl. No.: 11/152,660

(22) Filed: Jun. 14, 2005

(51) Int. Cl. A61K 9/48; A61K 31/522

(52) U.S. Cl. 424/439; 424/451; 514/263.34

(57) ABSTRACT

Encapsulated energy gel compositions are provided. The compositions include an energy gel center contained within an edible coating.

### TECHNICAL FIELD

Provided herein are encapsulated energy gel compositions, including encapsulated energy gel compositions containing one or more carbohydrates and/or one or more sources of protein.

### BACKGROUND

Energy gel compositions are gel-like compositions that are often used to help replenish fluids and enhance athletic performance. Energy gels are often packaged in plastic pouches or packets. Some athletes, however, find it both cumbersome and detrimental to athletic performance to tear open an energy gel packet and squeeze the gel into their mouths while maintaining a competitive pace at e.g., running, bicycling, skating, climbing, or roller-blading. The gel can also overflow onto an athlete's hands or athletic apparel, resulting in stickiness and/or stains. In addition, after use, an athlete often must either carry the sticky gel packaging, e.g., in his hand or a pocket, or find a receptacle to dispose of it.

### SUMMARY

Encapsulated energy gel compositions are provided. An encapsulated energy gel composition includes an energy gel composition coated with an edible coating. The edible coating facilitates portability and ease of use of the energy gel compositions.

An encapsulated energy gel composition includes an energy gel composition contained within an edible coating. The encapsulated energy gel composition can have a spherical shape, or an oblong shape, or an oval shape.

In one embodiment, an energy gel composition can include one or more carbohydrates. The one or more carbohydrates can include maltodextrin. The energy gel composition can further comprise caffeine. The energy gel composition can further include one or more cation sources, and/or one or more amino acids (e.g., branched chain amino acids), and/or one or more sources of protein. A ratio of total carbohydrates to total protein in the energy gel composition can be from about 1:1 to about 10:1, or from about 1:1 to about 5:1.

Another embodiment of an energy gel composition can include one or more protein sources. The one or more protein sources can include whey protein. The energy gel composition can further include one or more carbohydrates. A ratio of total protein to total carbohydrates in the energy gel composition can be from about 1:1 to about 10:1, or from about 1:1 to about 5:1. The energy gel composition can further comprise one or more amino acids (e.g., branched chain amino acids). The energy gel composition can further comprise caffeine. The energy gel can further include one or more cation sources.

In another embodiment, an energy gel composition includes caffeine and one or more cation sources. The energy gel composition can further include one or more gelling agents. The energy gel composition can include one or more sources of protein, and/or one or more carbohydrates, and/or one or more amino acids (e.g., branched chain amino acids).

The edible coating can comprise less than 20% by weight, or less than 10% by weight, of the encapsulated energy gel composition. The edible coating can include one or more sweeteners and one or more binding agents. The edible coating can include one or more emulsifiers. The edible coating can include one or more film formers. The edible coating can include one or more starches. In certain embodiments, the edible coating can be a hard shell capsule, soft shell capsule, or gel cap capsule.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patents, patent applications, and other references mentioned herein are incorporated by reference in their entirety. In case of conflict, the present specification, including definitions, will control. In addition, the materials, methods, and examples are illustrative only and not meant to be limiting.

The details of one or more embodiments of the invention are set forth in the accompanying drawings and the description below. Other features, objects, and advantages of the invention will be apparent from the description and drawings, and from the claims.

### DETAILED DESCRIPTION

Energy gels provide a quick source of energy and can, in some cases, restore electrolyte balance to athletes. One drawback to the use of energy gels, however, is their difficulty of use during athletic performance. Energy gel compositions that are coated with either hard or soft (e.g., gel cap or soft gel) coatings can allow an athlete or consumer to carry the encapsulated compositions in e.g., his pocket or a dispenser, without the need to dispose of unnecessary plastic packaging. The encapsulated energy gel compositions described herein include an energy gel composition, e.g., an energy gel center, surrounded by an edible coating. Kits containing the encapsulated energy gel compositions, as well as methods of using the encapsulated energy gel compositions are also provided.

Energy Gel Compositions

Energy gel compositions are useful for a variety of reasons, e.g., increasing available energy during exercise, replenishing depleted electrolytes, promoting fluid retention during exercise, or facilitating intestinal reabsorption of fluids during exercise; see e.g., U.S. Publication 2005/0095271, incorporated by reference herein. For example, energy gels can provide an easier to digest source of carbohydrates than solid energy compositions, such as energy bars or tablets.

Any energy gel can be encapsulated to provide the described encapsulated energy gel compositions. Typically, an energy gel composition for inclusion in the encapsulated compositions is a composition in gel form. In some cases, an energy gel composition includes one or more carbohydrates. In some cases, an energy gel composition includes one or more sources of protein. In certain cases, an energy gel composition for inclusion in the encapsulated compositions includes one or more carbohydrates and one or more sources of protein. Optional ingredients can include one or more sources of cations (e.g., sodium cations, potassium cations, or magnesium cations), caffeine, one or more vitamins (e.g., Vitamins C, A, B6, and E), and one or more amino acids (e.g., branched chain amino acids).

One embodiment of an energy gel composition can include one or more carbohydrates at a total weight percent of from about 55% to about 95%; such an embodiment can be referred to as a “high carbohydrate energy gel composition.” In such an embodiment, the one or more carbohydrates provide a large portion of the calories of an energy gel composition, e.g., from about 50% to about 100% of the calories of the composition, or any range therebetween, e.g., about 50% to about 65%; about 55% to about 65%; about 65% to about 75%; about 65% to about 85%; about 70% to about 90%; or about 80% to about 100% of the calories. As indicated, in a high carbohydrate energy gel composition, the one or more carbohydrates can be from about 55% to about 95% by weight of an energy gel composition, or any range therebetween. For example, an energy gel composition can be about 55% to about 65%; about 60% to about 70%; about 70% to about 75%; about 80% to about 85%; or about 90% to about 95% by weight one or more carbohydrates. A high carbohydrate energy gel composition can optionally include, independently, one or more vitamins, one or more amino acids, caffeine, one or more sources of cations, and one or more sources of protein.

Another embodiment of an energy gel composition can include less than 55% total carbohydrates by weight; such an embodiment may be referred to as a “low carbohydrate energy gel composition.” In certain cases, a low carbohydrate energy gel composition includes less than 40%, or less than 30%, or less than 20%, or less than 10%, or less than 5% total carbohydrates by weight. In certain embodiments, a low carbohydrate energy gel composition includes 0% total carbohydrates by weight. A low carbohydrate energy gel composition can optionally include, independently, one or more vitamins, one or more sources of cations, caffeine, one or more amino acids, and one or more sources of protein. For example, some embodiments of low carbohydrate energy gel compositions can include from about 10% to about 95% total protein by weight.

Another embodiment of an energy gel composition includes one or more protein sources in an amount of from about 10% to about 95% total protein by weight; such an embodiment may be referred to as a “recovery energy gel composition.” For example, certain embodiments of a recovery energy gel composition include from about 50% to about 80% total protein by weight; other embodiments include from about 30% to about 70% total protein by weight. Optional ingredients in a recovery energy gel composition include, independently, one or more vitamins, one or more amino acids, caffeine, one or more sources of cations, and one or more carbohydrates.

In certain cases where an energy gel composition includes one or more carbohydrates and one or more sources of protein, a ratio of total carbohydrate to total protein can range from about 10:1 to about 1:10, or any ratio therebetween. For example, a ratio of total carbohydrate to total protein can be about 9:1, or 8:1, or 7:1, or 6:1, or 5:1, or 4:1, or 3:1, or 2:1. In other cases, a ratio of total carbohydrate to total protein can be about 1:2, or 1:3, or 1:4, or 1:5, or 1:6, or 1:7, or 1:8, or 1:9.

In certain embodiments, an energy gel composition can include caffeine and one or more sources of cations; such an embodiment can be referred to as a “stimulant/electrolyte energy gel composition.” Optional ingredients include, independently, one or more vitamins, one or more amino acids, one or more carbohydrates, and one or more sources of protein.

If included in the energy gel compositions referred to above, the one or more carbohydrates can include simple or complex carbohydrates, or mixtures thereof. Simple carbohydrates include sucrose, glucose, galactose, dextrose, fructose, trehalose, lactose, and maltose. Complex carbohydrates include maltodextrin and other polysaccharides, including long chain polysaccharides. The one or more carbohydrates can also include one or more of the following: rice syrup, honey, corn syrup, high fructose corn syrup, molasses, maple syrup, beet sugar, cane sugar, and sucanat. In some cases, an energy gel composition can be 100% complex carbohydrates. In other cases, both complex and simple carbohydrates are included. A ratio of complex to simple carbohydrates can be in the range of from about 10:1 to about 1:1, or any value therebetween. For example, a ratio of complex to simple carbohydrates can be about 9:1, or 8:1, or 7:1, or 6:1, or 5:1, or 4:1, or 3:1, or 2:1. In certain cases, the ratio of complex to simple carbohydrates can be from about 7:1 to about 5:1.

A liquid can be included in the energy gel composition for, e.g., solubilization of dry ingredients. A liquid can be water, juice, coffee, espresso, or tea (e.g., chamomile tea or green tea). The amount of liquid will vary depending on the types of other ingredients used. For example, if a liquid source of carbohydrates is used, the volume of another liquid such as water may be reduced. The desired viscosity of the final gel solution will also affect the amount of liquid employed.

A source of protein can be a protein fraction derived from any vegetable or animal source. Examples include whey protein, soy protein, rice protein, pea protein, beef protein, chicken protein, egg protein, and fish protein. Protein sources are available commercially.

Caffeine can be included in an energy gel composition. Caffeine can be provided as a pure compound, or can be provided in the form of coffee, espresso, powdered coffee, powdered espresso, green tea, green tea extract, gotu kola and kola nut (and extracts), guarana or Brazilian cocoa, mate´, etc. Combinations of caffeine sources can also be included. Typically, the total amount of caffeine in a 1 oz (30 g) serving of an energy gel composition (e.g., an unencapsulated energy gel composition) can be about 0 to about 50 mg, or any value therebetween (e.g., about 5, 10, 15, 20, 25, 30, 35, 40, 45, 50 mg caffeine/1 oz energy gel).

One or more cation (e.g., electrolytes such as Na+, K+, Mg2+) sources can also be included in the energy gel composition, e.g., to replenish electrolytes lost through sweat. Typically, sodium, potassium, and magnesium salts that are safe for ingestion are used as the cation sources. For example, sodium citrate and potassium citrate can be used as sources of sodium and potassium cations, respectively. In certain embodiments, a sodium salt is used, or a sodium and potassium salt. The total amount of cation salts included can be from about 0.5% to about 3% by weight of the energy gel composition.

One or more amino acids can optionally be included in an energy gel composition. Any of the naturally occurring and/or essential amino acids can be included in an energy gel composition. In certain cases, one or more branched-chain amino acids such as leucine, valine, isoleucine, and histidine are included. In some cases, taurine can be included in an energy gel composition. The total amount of amino acids can range from about 0.0% to about 0.5% by weight of the energy gel composition, or any range therebetween, e.g., about 0.05% to about 0.1%; about 0.075% to about 0.15%; about 0.1% to about 0.25%; about 0.2% to about 0.3%; about 0.1% to about 0.3%; about 0.25% to about 0.4%; and about 0.35% to about 0.5%. Amino acids are widely available commercially.

One or more nutraceutical or herbal supplements can also be included in an energy gel, including, without limitation, glucosamine, chondroitan sulfate, ginkgo biloba, ginseng, ginger, blue green algae, curcumin, Echinacea, lobelia, papaya fruit powder, St. John's wort, wheat grass, ribose, and creatine.

An energy gel composition can include one or more natural or artificial flavorants. Edible natural and artificial flavorants are available commercially and in a wide variety of flavors; see additional disclosure, below. The amount of flavorants included in an energy gel composition will vary with the type of flavor, its intensity and concentration, consumer acceptance, etc. Flavors can be, but are not limited to, apple, cherry, strawberry, banana, orange, lime, pineapple, lemon, blueberry, raspberry, caramel, coffee, vanilla, mint, mango, peach, tangerine, watermelon, cinnamon, chocolate, grape, and mixtures thereof. Artificial sweeteners can also be included, such as saccharin, thaumatin, alitame, saccharin, aspartame, sucralose, and acesulfame K.

One or more vitamins can also be included in an energy gel composition, such as, without limitation, vitamins A, E, C, and B6. Vitamins C and E can provide anti-oxidant benefits. The total amount of vitamins included in an energy gel can range from about 0.00% by weight to about 0.5%, or any range therebetween, e.g., about 0.05% to about 0.1%; about 0.1% to about 0.18%; about 0.08% to about 0.18%; about 0.12% to about 0.22%; about 0.2% to about 0.35%; about 0.3% to about 0.4%; and about 0.38% to about 0.5% by weight of an energy gel composition.

One or more preservatives and acidity regulators that are safe for ingestion can also be included, e.g., sodium benzoate, potassium sorbate, citric acid, etc. The amount of such compounds will vary with the type of energy gel, but can range from about 0.0% to about 1.5% by weight, e.g., about 0.1% to about 0.5%; about 0.1% to about 1%; and about 0.5% to about 1.4%. Colorants, such as food quality dyes, can optionally be included and are commercially available.

An energy gel composition can include one or more gelling agents. A gelling agent can be, without limitation, gelatin, agar, carrageenan, locust bean gum, guar gum, xanthan, tragacanth, ghatti gum, pectin, and alginate.

Energy gel compositions can be prepared using methods known to those having ordinary skill in the art. Typically, the ingredients are mixed and heated, e.g., to about 160 to about 180° F.; see, e.g., U.S. Patent Publication 2005/0095271. After preparation, the energy gel compositions are coated, as described below.

Edible Coatings

The energy gel compositions described previously are coated to facilitate portability of the compositions and to reduce unnecessary packaging. The term “coated,” as used herein, means that an energy gel composition is substantially encompassed by, or contained within, a coating. For example, an energy gel composition can be completely encompassed by an edible coating.

A coating should contain edible ingredients that are safe for consumption and that are not considered to be detrimental to athletic performance. A coating can contain ingredients such as: one or more flavoring agents, one or more natural or artificial sweeteners, one or more dispersing agents, one or more coloring agents, one or more film formers, one or more emulsifiers, one or more anti-foaming agents, and one or more binding agents.

Flavoring agents include those commonly known in the art, such as essential oils and natural and synthetic flavors, or mixtures thereof, including but not limited to chocolate, vanilla, cinnamon, oils derived from plants and fruits, such as citrus oils (e.g., lemon, lime, orange, tangerine), fruit essences (e.g., banana, raspberry, cherry, strawberry, blueberry, mango, peach, pineapple), peppermint oil, spearmint oil, other mint oils, clove oils, oil of wintergreen, anise, menthol, and the like.

Natural and artificial sweeteners contemplated for the use in the coating include, but are not limited to, one or more of the following: sugar, corn syrup, confectioner's glaze, sucrose, fructose, dextrose, glucose, sorbitol, maltitol, xylitol, isomalt, other crystallisable polyols, trehalose, saccharin, thaumatin, alitame, saccharin, aspartame, sucralose, and acesulfame K.

Emulsifiers can be included in an edible coating. Examples of emulsifiers are, without limitation, lecithin, sodium stearoyl lactylate, calcium stearoyl lactylate, lactylated monodiglycerides, polyglycerol esters, and mono-diglycerides.

An edible coating can include coloring agents. Coloring agents include food quality dyes. Film formers can also be included in an edible coating, such as, without limitation, methylcellulose, gelatins, hydroxpropyl cellulose, hydroxyethyl cellulose, ethyl cellulose, carboxymethyl cellulose, and mixtures thereof. Binding agents may be included, such as gum arabic, alginate, cellulosic vegetable gums, vegetable fibers, and the like, e.g., as described above for gelling agents.

Standard coating procedures and formulations used in, e.g., the confectionary and pharmaceutical arts can be used to coat energy gel compositions. See, e.g., U.S. Pat. No. 5,536,511 and WO 99/44436.

An edible coating can be in the form of an edible capsule. Edible capsules include, for example, a gel cap, a soft shell capsule (e.g., soft gel capsule), or a hard shell capsule (e.g., hard gel capsule). The major difference between a hard shell capsule and a soft shell capsule or gel cap is that a soft shell or gel cap is physically a softer container. Thus, for example, an energy gel composition described herein can be encapsulated by a soft gel capsule. In other cases, a hard shell capsule may be preferred, e.g., for use in sporting events where greater physical stability of the compositions to rupture may be required.

Hard shell capsules, soft shell capsules (e.g., soft gels), and gel caps are composed of an outer material or casing which is safe for ingestion and typically dissolves after ingestion by the consumer, with the interior portion of the capsule being filled with the energy gel composition. Capsules can be prepared from gelatin, e.g., a bovine or pig source of gelatin, or from vegetable starches, e.g., food starches, such as potato starch. Capsules can also include beeswax, carnauba wax, and other polymeric media, in addition to other ingredients described previously, such as sweeteners, emulsifiers, and binding agents.

Capsules can be made using a variety of conventional means and formulations known to those having ordinary skill in the art. For example, soft gel capsules can be prepared by conventional means, e.g., as described in U.S. Pat. No. 6,333,047.

An edible coating can be less than 40% by weight of an encapsulated energy gel composition. For example, an edible coating can be less than 20% by weight of an encapsulated energy gel composition, or less than 10% by weight of an encapsulated energy gel composition, or less than 5% by weight of an encapsulated energy gel composition.

Encapsulated energy gel compositions can be provided in sizes and shapes that are acceptable for the consumer or athlete to chew and swallow. Encapsulated energy gel compositions can be any size, e.g., ranging from less than a single serving size up to a suggested single serving size, or more. Typically, an athlete can consume a certain number of encapsulated energy gel compositions to result in a suggested serving size of the energy gel composition itself, e.g., 1, 2, 3, 4, 5, 6, 7, 8, 9, 10, or more encapsulated energy gel compositions to result in a suggested serving size of the energy gel composition. For example, in one embodiment, a serving size of encapsulated energy gel compositions will provide about 1 oz (about 28 g) of the energy gel composition itself. In other cases, a serving size of encapsulated energy gel compositions will provide about 2 oz (about 57 g) of the energy gel composition itself. In yet other cases, a serving size of encapsulated energy gel compositions will provide about 3 oz (about 85 g) of the energy gel composition itself. An encapsulated energy gel composition is typically in the range from about 5 g to about 35 g in weight, or any size therebetween (e.g., about 5, 6, 7, 8, 9, 10, 11, 12, 13, 14, 15, 20, 25, 30, 32, or 34 g in weight). In certain embodiments, an encapsulated energy gel composition is from about 5 g to about 10 g in weight.

Encapsulated energy gel compositions can be provided in any shape, including spherical, oblong, and oval. In certain embodiments, an encapsulated energy gel composition is spherical in shape. In other embodiments, an encapsulated energy gel composition is oval in shape.

Once coated, one or more encapsulated energy gel compositions can be placed in packaging typically employed for e.g., edible compositions or medicines, such as plastic, poly-foil, film, or foil wrappings, sheets, tubes, pouches, boxes, containers, etc. Depending on the needs and desires of the athlete or consumer, he can then carry the above-described encapsulated energy gel compositions in their original packaging or can unwrap them for placement, e.g., in his pocket.

In certain cases, an athlete or consumer can employ a dispenser to carry and dispense the encapsulated energy gel compositions. A dispenser can be adapted to be attached to e.g., a belt, belt clip, sleeve, shorts or pants, wristband, heart monitor, GPS monitor, or bicycle handlebars or frame. A dispenser can be made of moldable plastic.

Kits

Also provided herein are kits. A kit can include one or more encapsulated energy gel compositions and associated packaging (e.g., a box, pouch, bag, etc.). One or more optional ingredients can also be included, such as: energy bars, energy drinks, fluid/electrolyte replacement drinks, recovery/replenishment drinks, and/or a dispenser for the encapsulated energy gel compositions. The one or more encapsulated energy gel compositions can be provided in a variety of flavors and packaging modes for flexibility of use and storage by the consumer or athlete.

Methods of Use

Methods for using the encapsulated energy gel compositions, e.g., to replenish electrolytes, to provide energy, to prevent hyponatremia during exercise, or to promote muscle recovery after exercise are also provided. The methods involve consuming an encapsulated energy gel composition, e.g., either before, during, or after athletic activity. Consumption of an encapsulated energy gel compositions can be preceded with or followed by consumption of water or a fluid/electrolyte replacement drink, depending on the nature of the encapsulated energy gel composition ingested. For example, if the encapsulated energy gel composition contains electrolytes such as sodium, then typically the consumer will not want to consume additional electrolytes in the form of an electrolyte replacement drink. If the encapsulated energy gel composition does not contain electrolytes, the consumer may want to consume electrolytes in the form of an electrolyte replacement drink. Typically, a consumer should drink approximately 8 oz.-16 oz. of fluid per 1 oz of unencapsulated energy gel composition.

A number of embodiments of the invention have been described. Nevertheless, it will be understood that various modifications may be made without departing from the spirit and scope of the invention. Accordingly, other embodiments are within the scope of the following claims.

What is claimed is:

1. An encapsulated energy gel composition comprising an energy gel composition contained within an edible coating.

2. The composition of claim 1, wherein the edible coating is a hard shell capsule, soft shell capsule, or gel cap capsule.

3. The encapsulated energy gel composition of claim 1, wherein the edible coating comprises less than 20% by weight of the encapsulated energy gel composition.

4. The encapsulated energy gel composition of claim 1, wherein the edible coating comprises less than 10% by weight of the encapsulated energy gel compositions.

5. The encapsulated energy gel composition of claim 1, wherein the encapsulated energy gel composition has a spherical shape.

6. The encapsulated energy gel composition of claim 1, wherein the encapsulated energy gel composition has an oval shape.

7. The encapsulated energy gel composition of claim 1, wherein the edible coating comprises one or more sweeteners and one or more binding agents.

8. The encapsulated energy gel composition of claim 7, wherein the edible coating further comprises one or more emulsifiers.

9. The encapsulated energy gel composition of claim 7, wherein the edible coating further comprises one or more film formers.

10. The encapsulated energy gel composition of claim 7, wherein the edible coating further comprises one or more starches.

11. The encapsulated energy gel composition of claim 7, wherein the edible coating further comprises one or more dispersing agents.

12. The encapsulated energy gel composition of claim 7, wherein the edible coating further comprises one or more flavoring agents.

13. The encapsulated energy gel composition of claim 1, wherein the energy gel composition comprises one or more carbohydrates.

14. The encapsulated energy gel composition of claim 13, wherein the one or more carbohydrates comprise maltodextrin.

15. The encapsulated energy gel composition of claim 13, wherein the energy gel composition further comprises caffeine.

16. The encapsulated energy gel composition of claim 13, wherein the energy gel composition further comprises one or more cation sources.

17. The encapsulated energy gel composition of claim 13, wherein the energy gel composition further comprises one or more amino acids.

18. The encapsulated energy gel composition of claim 17, wherein said one or more amino acids comprise a branched chain amino acid.

19. The encapsulated energy gel composition of claim 13, wherein the energy gel composition further comprises one or more sources of protein.

20. The encapsulated energy gel composition of claim 19, wherein a ratio of total carbohydrates to total protein in the energy gel composition is from about 1:1 to about 10:1.

21. The encapsulated energy gel composition of claim 1, wherein the energy gel composition comprises one or more protein sources.

22. The encapsulated energy gel composition of claim 21, wherein the one or more protein sources comprise whey protein.

23. The encapsulated energy gel composition of claim 21, wherein the energy gel composition further comprises one or more carbohydrates.

24. The encapsulated energy gel composition of claim 23, wherein a ratio of total protein to total carbohydrates in the energy gel composition is from about 1:1 to about 10:1.

25. The encapsulated energy gel composition of claim 21, wherein the energy gel composition further comprises one or more amino acids.

26. The encapsulated energy gel composition of claim 25, wherein the one or more amino acids comprise a branched chain amino acid.

27. The encapsulated energy gel composition of claim 21, wherein the energy gel composition further comprises caffeine.

28. The encapsulated energy gel composition of claim 21, wherein the energy gel further comprises one or more cation sources.

29. The encapsulated energy gel composition of claim 1, wherein the energy gel composition comprises caffeine and one or more cation sources.

30. The encapsulated energy gel composition of claim 29, wherein the energy gel composition further comprises one or more gelling agents.
